(19) United States
(12) Patent Application Publication
(10) Pub. No.: US 2007/0199890 A1
(43) Pub. Date: Aug. 30, 2007

(54) Antimicrobial activated carbon and use thereof

(75) Inventor: Jeffrey A. Trogolo, Boston, MA (US)

Correspondence Address: EDWARD K. WELCH II;IP&L Solution, 4558 Ashton Court, Naples, FL, 34112, US

(73) Assignee: AgION Technologies, Inc.

(21) Appl. No.: 11/362,932

(22) Filed: Feb. 27, 2006

(51) Int. Cl. B01D 39/14; A61K 33/44

(52) U.S. Cl. 210/500.1; 424/125; 427/2.24

(57) ABSTRACT

Antimicrobial activated carbon having controlled release of the antimicrobial active agent are provided comprising activated carbon and an antimicrobial coating applied to at least a portion of its exposed outer surface wherein the antimicrobial coating comprises a binder and an antimicrobial water soluble glass or an inorganic ion exchange type antimicrobial agent. The antimicrobial coatings to be applied to the activated carbon materials are curable liquids or ultra-fine thermoplastic powder coatings. The antimicrobial activated carbon materials may be employed in filters as a loose fill or in sintered filters.

### BACKGROUND

1. Technical Field

The present invention relates to a novel material useful as an antimicrobial composition, to antimicrobial filtration media prepared from the same and a method for preparing the foregoing. The invention particularly relates to antimicrobial activated carbon wherein the antimicrobial characteristics arise as a result of the treatment of the activated carbon with antimicrobial water soluble glass or antimicrobial inorganic ion exchange materials, both of which contain and/or are capable of releasing one or more antimicrobial metal ions, which produce the antimicrobial effect.

2. Description of the Prior Art

Activated carbon is a well-established material for use as a liquid phase and gas phase adsorbent to remove impurities from liquids and gases. Activated carbon is also used to recover specific desirable compounds from process and waste chemical streams via the process of adsorptive separation. Activated carbon is routinely utilized in the purification of potable drinking water and for the removal of residual contaminants in wastewater treatment processes. Activated carbon is used in industrial processes and automotive air pollution control systems to recover solvents and gasoline. Regenerative and single use activated carbon systems and activated carbon filled canisters are used to remove contaminants in vapor exhaust streams; these systems may serve as the primary step in removal of exhaust stream contaminants or as the final polishing step. Activated carbon for liquid phase applications generally have a pore size of greater than 3 nm whereas the materials used in gas phase applications generally have a pore size of less than 3 nm. Activated carbon is routinely used to remove odors, color, taste and other objectionable impurities by trapping these undesirable compounds. (See Soffel, R. W, “Carbon, Activated Carbon” Kirk-Othmer Concise Encyclopedia of Chemical Technology, Abridged Version of the 24 volume Encyclopedia of Chemical Technology, 3^{rd}. Ed., pp. 204-205)

Despite the attributes of activated carbon filters, a problem that has long plagued them is the growth of bacteria in the filter media, especially filter media employed in aqueous or water containing applications and processes or exposed to high humidity that may be absorbed by the filter. Bacteria may be introduced to the filter media during manufacture and/or packaging and is certainly introduced during use. The former arises as a result of the use of contaminated water during the wetting of the filter media or poor hygiene and/or handling during the manufacture and packaging operations. For example, activated carbon water filters are typically packaged, stored and shipped in a pre-moistened state. Alternatively, bacteria in water or other liquids being treated tends to deposit on or is trapped by the activated carbon or bacteria may be introduced to the filter during installation, maintenance, etc., by, e.g., hand contact. Given the moist conditions and the presence of organic matter trapped or adsorbed by the activated carbon, such filter media essentially serve as incubators for the growth and proliferation of bacteria and, consequently, biofilms. Furthermore, given the rapid rate at which bacteria multiply, even the period of time between intermittent uses, especially as seen with carafe type water filter systems, is oftentimes sufficient to enable substantial growth in the number and size of bacterial colonies. Certainly, the time between manufacture and use provides more than enough time for bacteria to wreak their damage on a pre-moistened filter.

While contaminated newly manufactured filter media tend to give off a distinct odor and/or are discolored upon opening their packaging, contamination in filters in use is oftentimes not to a level that detection is readily noticeable. Regardless, when a contaminated newly manufactured filter is first used or when a filter is reused following a period of inactivity, the initial flow of water passing through the filter media becomes contaminated with the bacteria. Consequently, the user may notice a telltale taste in or an odor arising from the water being consumed or, worse, suffer the associated health effects, especially gastrointestinal problems or discomfort.

Besides the aforementioned problems, even in the absence of any health problems, the presence of bacteria and biofilms tends to have an adverse effect on the purification performance of the activated carbon. Specifically, bacteria and biofilms tend to clog the pores as well as serve as a barrier between the surface of the activated carbon and the water it is intended to filter. The blockage of an individual pore or blockage within a pore channel causes a markedly disproportionate loss of surface area available for adsorbing water contaminants since the far greater surface area of the activated carbon is internal, which is accessed through the pores: the exposed outer surface accounting for just a small percentage, if not fraction of a percent, of the total “effective” surface area of the activated carbon material. Thus, as more and more pores and, consequently, surface area is covered by or blocked by the bacteria and/or biofilm, the performance capability of the activated carbon is greatly reduced.

In an effort to overcome these problems, Piccione et. al., U.S. Pat. No. 3,294,572, impregnated activated carbon with silver by treating the activated carbon with an acid solution of a silver salt and subsequently subjecting the so treated material to high heat treatment, above 450° C., for a sufficient time to cause metallic silver to deposit on the activated carbon surface. This deposition occurs on both the exposed outer surface and the unexposed inner surface of the pore channels or tunnels. By this process, the patentee claims to have achieved loadings of up to 70% of silver based on the weight of the carbon.

The success of these silverized activated carbons was not without problems. As more silver is deposited, presumably in an effort to increase its antimicrobial efficacy, its water purification abilities, i.e., its ability to remove impurities from the water, decrease markedly even at levels as low as 1% silver. It is believed that this results, in part, from the silver itself blocking access to the pores as well as, more importantly, depositing on the inner surface to such an extent that the silver physically blocks or severely restricts the flow of materials through the channels or tunnels in the activated carbon. Consequently, as noted above, the increased deposition of silver reduces the efficacy of the activated carbon. Due to this marked effect, commercial silverized activated carbon, especially that intended for water filters, tends to have a silver content of only about 0.1 wt. percent.

However, silver deposits are not the only factor adversely affecting performance of the silverized activate carbon. During use, deposits of materials adsorbed from the water being treated as well as dead bacteria build up on and within the activated carbon material blocking pores, blocking or restricting flow within the channels or tunnels, and creating a barrier between the activated carbon surface and the water to be treated. Thus, again, performance falls off markedly as more and more water is filtered and/or as its in-service time lengthens.

In an effort to lessen these effects, Mitsumori et. al., U.S. Pat. No. 4,045,553, developed a process to reactivate spent filters or filters having diminished performance by subjecting the spent activated carbon to steam. Though believed efficacious, such a process is not practical for the commercial, and certainly not the consumer, setting.

Another route to addressing the adverse consequence of silver on activated carbon while maintaining the antimicrobial capabilities of the filter media was to add the antimicrobial agent as a separate component, generally as part of another material or additive to the compositional make-up of the filter media. For example, Beauman et. al., U.S. Pat. No. 4,396,512, proposed the preparation of a filter media mixture comprised of a specially treated, dried, silver-bearing, highly purified, inert material in particulate or fibrous form, such as cellulose fibers, mixed with specific proportions of powdered, activated carbon filter material. Bacterial growth in and on the filter media is inhibited by silver ions that slowly eluted or dissolved from the segregated, silver-treated cellulose fibers uniformly interspersed among the activated carbon filter material. While this addressed concern with silver precipitate blocking the pores and creating a barrier between the water to be treated and the activated carbon, it results in an overall reduction in the amount of activated carbon present in a filter of a given size. Thus, avoiding the direct impact on the activated carbon nevertheless causes an overall reduction in the performance and longevity of the filter due to the reduction in activated carbon content.

Another factor influencing the use of silver in water filters, one unrelated to performance or whether the silver is present on the activated carbon or another carrier, is the regulatory controls on the permissible level of silver and silver ions in potable water. In the United States, water contaminants are regulated by the US Environmental Protection Agency which has established a Secondary Maximum Contaminant Level (SMCL) for silver of 0.1 mg silver per liter of water. More stringent guidelines have been established by the World Health Organization (WHO), which recommends that the level of silver in drinking water not exceed 0.05 mg per liter.

A number of factors influence the level or amount of silver released into the water being treated by a given filter element. For example, the overall amount of silver or silver source present will play a large role; however, an even greater role is played by the form in which the silver is present in the filter media, both from a chemical as well as a physical standpoint. Specifically, both of these factors greatly influence the solubility and rate of dissolution of silver or the silver source in water: characteristics that also determine the useful life of the filter, at least from an antimicrobial perspective. As is well known, silver metal itself is poorly soluble and of limited antimicrobial bioefficacy: the much more active and efficacious species of silver being the silver ion. Thus, to provide excellent solubility and antimicrobial performance, it is necessary to convert the silver metal to a salt. The most typical process for this conversion occurs naturally and involves the oxidation at the metal surface, and subsequent dissolution of the silver oxide. Many silver salts, such as silver oxide, are readily soluble: whereas others, such as silver chloride, have low solubility. Thus, environmental conditions which promote the production of one salt over another from silver metal will also influence the release of antimicrobial silver ions and, thus, the bioefficacy of the antimicrobial agent.

Another factor influencing the release of the silver ions is the physical form of the silver source. As is known through the Gibbs-Thompson effect, surfaces with small radii of curvature have higher solubility than the corresponding flat surface. In silverized activated carbon as well as in other silverized carriers, the silver precipitates or deposits in the form of small, nano-sized, silver particles or nodules. Thus, at least initially, the silver particles in silverized GAC and other silverized carriers have a high, uncontrolled solubility. As more and more silver is dissolved, the particles or nodules flatten out whereby silver release drops to low, though, at least for a while, efficacious levels: ones acceptable under current regulatory schemes. Even though the amount of silver released will eventually drop quite significantly, the problem lies with the initial utilization of these water filters as well as their use following pronged periods of inactivity, especially in the early life of the water filter where there is still some radii of curvature to the silver particles or nodules. Oftentimes, in these circumstances, the amount of silver released into the water exceeds that which is allowed under the safe drinking water standards.

In order to address this problem it is necessary to flush the filters in order to remove the excess silver before putting the filter in use or saving the filtered water for consumption. Indeed, the instructions for commercial filters employed with carafes for home use instruct the user to discard the first several carafes of purified water before placing the carafe into everyday use. With in-line filters, one is instructed to allow the water to run for a minimum period of time to ensure that the excess silver is flushed from the filter media. For in-line filters that are used intermittently, as with a filter attached to one's faucet, again flushing and discarding of the initial filtrate is advised.

While flushing is an easy fix to avoid the consumption of high levels of silver, this overlooks a much more disconcerting problem in that the rapid and initially large dissolution and subsequent expulsion of silver shortens the overall life expectancy of the filter, at least from a bioefficacy standpoint. Specifically, the initial burst or release if silver ions as well as the higher dissolution rate during the early life of the filter means that a substantial amount of the antimicrobial agent is lost early in the lifetime of the filter media. Furthermore, following on the discussion above regarding the Gibbs-Thompson effect, as the silver particles or nodules on the activated carbon surface dissolve, they flatten out, thereby reducing the rate of silver oxidation and, consequently, silver release. Since there must be a certain level of silver release in order for bioefficacy to manifest, too slow and/or too little silver release and antimicrobial properties will not be seen. Consequently, as silver release drops, the efficacy of the antimicrobial filter also falls off leading to biofilm buildup in the filter, increased pressure drop and, consequently, shortened lifetime and frequent changes in filter material. This rapid release of silver coupled with the drinking water regulatory limits for silver therefore has necessitated the design of filters with a more controlled performance.

Many efforts have been undertaken to reduce or eliminate this rapid release of silver or silver ions, thereby preserving longevity. For example, Adachi et. al., U.S. Pat. No. 5,342,528, employ bone-char in combination with a water purifying material comprising an activated carbon having (a) silver and/or an inorganic silver compound and (b) a water-soluble alkaline earth metal salt supported thereon. The bone char is said to regulate or affect the release of the silver ions from the metallic silver or inorganic silver compound, whichever is present. However, these compositions employ very low levels of silver, from about 0.05 to 0.5%. Furthermore, the presence of bone char means less activated carbon is present for a given volume of filter media. Thus, longevity is compromised nevertheless.

In yet another effort, Pimenov et. al., U.S. Pat. No. 6,514,413, disclose a method for disinfecting and purifying tap water and untreated water using a composite bactericidal adsorption material as a filter, said adsorption material comprising a substantially uniformly distributed admixture of granules of iodinated anion-exchange resin, granular activated carbon, a silver containing adsorbent and amphoteric fibers. The silver containing adsorbent comprises silver containing cation exchange resin or silver containing modified polyacrylonitrile based fibers. Unlike prior silver antimicrobial water filters which release silver ions into the water, Pimenov et. al. had found that the iodine ions of the iodinated ion-exchange resin, as well as free chloride ions in the water, reacted with the silver ions to form practically insoluble silver salts that precipitated onto the iodinated ion-exchange resin where they held the silver ions while concurrently blocking the release of the iodine ions.

While Pimenov et. al. avoid the concern with respect to the release and presence of silver ions in drinking water as well as, in part, the brevity of bioefficacy, the overall purification performance is compromised in that a filter of a given volume has less activated carbon due to the presence of the multitude of other components. More importantly, in Pimenov et. al. bioefficacy is reliant upon contact of the bacteria with the precipitated silver salts on the fibers. Since there is no release or movement of the silver ions, bacteria not in contact with the precipitated silver salts are not affected. Furthermore, the deposit or adsorption of any organic matter or the generation of a biofilm, particularly as a result of the initial deposit and killing of bacteria, on the surface area where the silver salts have precipitated will render them ineffective. In essence, the organic matter or biofilm will serve as a barrier between the bacteria and the silver salts.

Despite all the efforts to develop efficacious and long lived antimicrobial water filter and purification media and related means, none have achieved overall success in addressing silver release issues concurrent with providing efficacious and long lived antimicrobial properties.

Thus, there remains a need for antimicrobial filter media and filter elements, particularly water filter media and filter elements, that release an antimicrobial agent at levels that are commercially bioefficaceous without compromising the utility or suitability of the filter media for its intended end-use application. In particular, there remains a strong need for antimicrobial water filter media and filter elements, especially ones based on silver antimicrobial agents, which release antimicrobial metal ions, especially silver ions, at sufficient levels to provide bioefficacy without exceeding safe drinking water standards, initially as well as during use and/or following extended periods of inactivity.

Additionally, there remains a need for antimicrobial water filter media and filters having a controlled release of the antimicrobial active, especially silver ions, for enhanced longevity. Similarly, there remains a need for antimicrobial water filter media and filters having an essentially constant release of antimicrobial active throughout its useful life.

There also remains a need for antimicrobial water filter media and filters having an antimicrobial agent whose impact, if any, on the purification capabilities of activated carbon is insubstantial.

Finally, there remains a need for an antimicrobial water filter media where an antimicrobial agent can be employed without requiring a noticeable compromise on the amount or volume of activated carbon present for a given sized water filter.

### SUMMARY

The present invention relates to antimicrobial activated carbon materials, especially granular activated carbon (GAC), having excellent and long-lived antimicrobial properties without the concurrent reduction in purification properties of the activated carbon as typically seen with silverized activated carbon materials or with the replacement of a portion of the GAC with an antimicrobially treated additive or filler material. Specifically, the present invention is directed to activated carbon particles having applied thereto an antimicrobial coating composition comprising an antimicrobial agent in a binder material wherein the antimicrobial agent is an ion-exchange type antimicrobial agent or a dissolving glass type antimicrobial agent. Preferably, the antimicrobial agent is an ion-exchange type antimicrobial agent comprising ion-exchanged antimicrobial metal ions on a suitable carrier including zeolites, hydroxyapatites, and zirconium phosphates. The binder material is preferably selected from hydrophilic polymers, thermoset resins, thermoplastic polymers and inorganic binders such as silicates. The antimicrobial coating composition, as applied, is preferably of a suitable viscosity that the coating does not substantially enter the pores of the activated carbon particle and, most preferably, is of such low surface tension that it will not remain over the pores once it is applied and before it cures or sets.

In another embodiment, the present invention is directed to activated carbon particles which have been partially coated with an antimicrobial coating composition comprising the antimicrobial agent in a suitable binder wherein the viscosity and surface tension characteristics of the binder is not as critical since a portion of the activated carbon surface has no coating applied thereto so that the pores and internal structure of the activated carbon particle are accessible for providing the purification properties associated therewith. Preferably, in accordance with this aspect of the present invention, the exposed surface area of the activate carbon upon which the coating is applied is no more than 60%, preferably no more than 50%, most preferably no more than 40%, of the exposed surface area. While viscosity is not so critical in this aspect of the present invention, it is most preferable that the coating be of a sufficiently low viscosity and/or have such surface tension characteristics that the pores, or at least a substantial percentage thereof, on those portions of the activated carbon particles coated with the antimicrobial coating are not blocked by the coating composition.

In yet another embodiment of the present invention the antimicrobial coating applied to the activated carbon comprises a binder and particles of a hydrophilic polymer containing one or more, preferably a large plurality of, individual particles of the antimicrobial agent. These hydrophilic particles, also referred to as encapsulated antimicrobial agents, remain as discrete particles or phases in the binder, including where the binder is also a hydrophilic polymer.

The present invention also pertains to activated carbon particles having multiple layers of an antimicrobial coating applied thereto wherein those layers applied first have a higher concentration of the antimicrobial agent than the latter applied layers. In a preferred embodiment, each successive layer has less antimicrobial agent than the previous layer and, optionally, the outermost layer may be free of antimicrobial agent.

Another aspect of the present invention pertains to the method of making an antimicrobial activated carbon particle comprising the steps of applying an antimicrobial coating to at least a portion of the exposed surface area of the activated carbon particles, allowing the coating to cure or set and recovering the coated activated carbon particles. Alternatively, the present invention also pertains to the method whereby a plurality of layers of an antimicrobial coating is applied to the exposed surface of the activated carbon particles.

The present invention also pertains to a method of producing antimicrobial activated carbon particles which method comprises the steps of applying a binder coating to at least a portion of the activated carbon particles and then dusting the wetted activated carbon particles with particles of the antimicrobial agent before the binder coating cures or sets, allowing the binder coating to cure or set and then recovering the coated activated carbon particles. In yet another alternative, the antimicrobial agent may be carried in a mist of an activator solution for curing the binder composition.

Another aspect of the present invention pertains to water filters made using the aforementioned antimicrobial activated carbon particles. Although the water filter may comprise the antimicrobial activated carbon in a loose state, it is preferred that the water filter comprises the antimicrobial activated carbon in a sintered state.

Finally, the present invention also pertains to a method of making water filters using the aforementioned antimicrobial activated carbon materials wherein the filter media is present as a loose fill or as a sintered material.

### BRIEF DESCRIPTION OF THE DRAWINGS

FIG. 1 depicts a cross-sectional view of a portion of an antimicrobial activated carbon particle made in accordance with the present invention.

FIG. 2 is a scanning electron microscope photograph of a portion of an antimicrobial activated carbon particle made in accordance with the present invention.

FIG. 3 is a close up scanning electron microscope photograph of a portion of the antimicrobial activated carbon particle shown in FIG. 2.

FIG. 4 is a cross-sectional depiction of a section of a coated activated carbon particle having a plurality of layers of different antimicrobial concentration.

FIG. 5 is a graph showing the measured ion release profile of the prior art versus two different embodiments of the present invention.

### DETAILED DESCRIPTION OF THE PREFERRED EMBODIMENT

All patent applications, patents, patent publications and literature references cited in this specification are hereby incorporated by reference in their entirety. In the case of inconsistencies, the present description, including definitions, is intended to control.

The present invention is directed to activated carbon, especially granular activated carbon, which is treated with an antimicrobial agent. Essentially any activated carbon material may be employed in the practice of the present invention including those derived from lignite, coke, charcoal, coal, bones, wood, peat, petroleum byproducts and coconut shell. Such activated carbon materials typically have a surface area of from about 100-2,000 m²/g, a particle size of from about 50 to 3000 microns, and may be employed in a number of shapes including spheres, columns, crushed shapes, powder, fibrous and granules. As noted above, the activated carbon is preferably in the shape of granules and, for convenience, the discussion of the present invention is typically made in reference to the granular form. The specific selection of the activated carbon material will depend, in part, upon the intended end-use application for the antimicrobially treated activated carbon, especially the nature of the material to be subjected to activated carbon purification or treatment. For example, virgin activated carbon derived from bituminous coal, such as ARCE Systems 8×30 BC Granular Activated Carbon, is especially suited for liquid phase applications whereas ARCE Systems 12×40 Granular Activated Carbon is especially suited for gas-phase applications. Generally speaking, activated carbons are widely known and available from a multitude of commercial sources. Those skilled in the art will readily recognize the activate carbon to be selected for their specific application.

Antimicrobial coating compositions suitable for use in the practice of the present invention are curable compositions comprising either an ion-exchange type antimicrobial agent or a dissolving glass type antimicrobial agent and a binder material comprising a hydrophilic polymer, a thermoset resin, a thermoplastic polymer or an inorganic binder material, or the precursors for the same. As used herein and in the appended claims, the terms “curable”, “cure” or “set” refer to the ability or transformation of a liquid or a flowable 100% solids coating composition to a solid, finished coating. Most often the terms “curable”, “cure” and “cured” will be in reference to traditional thermoset or cross-linkable coating compositions wherein cure or polymerization/crosslinking is effectuated by any number of circumstances or conditions including as a result of the combination of reactive constituents and/or the exposure of the same to environmental conditions which effectuate cure, e.g., heat, actinic radiation (including UV light), moisture, etc. These terms as well as the term “set” are also used in relation to those coatings that form through solvent evaporation or a combination thereof with cross-linking. Finally, though not a traditional use of these terms, unless specifically stated otherwise, these terms also include powder coating applications wherein a fine powder of a thermoplastic carrying the antimicrobial agent is applied and heat fused to the exposed surface of the activated carbon particles.

As noted, the antimicrobial agent is selected from antimicrobial water soluble glasses or ion-exchange type antimicrobial agents wherein the antimicrobial agent active component is one or more antimicrobial metals, metal salts or metal ions, most preferably one or more antimicrobial metal ions. Suitable antimicrobial metals and metal ions include, but are not limited to, silver, copper, zinc, gold, mercury, tin, lead, iron, cobalt, nickel, manganese, arsenic, antimony, bismuth, barium, cadmium, chromium and thallium. Metal ions of silver, copper, zinc, and gold or combinations thereof are preferred because they are considered safe for in vivo use. Silver ions, alone or in combination with copper or zinc or both, are more preferred due to the fact that they have the highest ratio of efficacy to toxicity, i.e., high efficacy to low toxicity.

As noted, the antimicrobial agent may be in the form of an antimicrobial water soluble glass. These glasses typically comprise a water soluble borosilicate or phosphate glass containing antimicrobial metal containing compounds, preferably inorganic antimicrobial metal salts, especially antimicrobial metal oxides such as silver oxide, copper oxide, and the like. As the glass is dissolved, the antimicrobial metal containing compound, e.g., the metal oxide, dissociates releasing the antimicrobial metal ions. By suitable adjustment of the glass composition as well as the level of the antimicrobial agent contained therein, the dissolution rate of the glass in water and, consequently, the release rate of the antimicrobial metal ion can be controlled. Antimicrobial water soluble glasses suitable for use in the practice of the present invention are described in, e.g., U.S. Pat. Nos. 5,290,544; 5,766,611; 6,410,633; and U.S. Pat. No. 6,593,260; and Published US Patent Application number US 20010006987. A family of especially desirable antimicrobial water soluble glasses, one based on antimicrobial silver salts and/or ions, is commercially available from Ishizuka Glass Co., Ltd. and through its numerous distributors worldwide, under the IonPure brand name.

Preferably, the antimicrobial agent will be in the form of an ion-exchange type ceramic particle wherein antimicrobial metal ions have been exchanged (replaced) for other non-antimicrobially effective ions in the ceramic particles or a combination of the foregoing with an antimicrobial metal salt. Antimicrobial ceramic particles include, but are not limited to zeolites, hydroxyapatite, zirconium phosphates and other ion-exchange ceramics. Hydroxyapatite particles containing antimicrobial metals are described in, e.g., U.S. Pat. No. 5,009,898. Zirconium phosphates containing antimicrobial metals are described in, e.g., U.S. Pat. Nos. 5,296,238; 5,441,717 and U.S. Pat. No. 5,405,644. More preferably, the antimicrobial agent is an antimicrobial zeolite containing ion-exchanged antimicrobial metal ions. Antimicrobial zeolites, including the antimicrobial zeolites disclosed in U.S. Pat. Nos. 4,911,898; 4,911,899 and U.S. Pat. No. 4,938,958, are well known and may be prepared for use in the present invention using known methods. Though much of the discussion of the ion-exchange antimicrobial agents will be focused on the zeolites, those skilled in the art will recognize that the discussion, as well as the ranges, parameters, etc. mentioned, are equally applicable to and readily translatable to the other ion-exchange carriers. Furthermore, since all of these antimicrobial agents are commercially available and described in the patent literature, as mentioned above, their composition and the like are known to those skilled in the art.

Generally speaking, ion-exchange type antimicrobial agents are prepared by an ion-exchange reaction in which non-antimicrobial ions such as sodium ions, calcium ions, potassium ions and/or iron ions, present in the carrier particles are partially or wholly replaced with antimicrobial metal ions. Suitable antimicrobial metal ions include those mentioned above. Similarly, as noted previously, the preferred antimicrobial metal ions employed in the ion-exchange type antimicrobial agents are silver, copper and zinc ions or combinations thereof, and most preferably silver ions alone or together with one or both of the others. For example, a combination of silver and copper ions provides both the antibacterial properties of the silver ions and the antifungal properties of the copper ions. Thus, one is able to tailor the antimicrobial agent by selection of specific metal ions and combinations thereof to be incorporated into the ion-exchange carrier particles for particular end-use applications.

In addition, other compounds may be used in combination with or, preferably, other ions may be exchanged into the carrier particles for the purpose of imparting better efficacy and/or color stability to the antimicrobial agents. For example, certain salts, such as sodium salts, including sodium nitrate, may be used in combination with the ion-exchange antimicrobial agent to enhance the initial release of the antimicrobial agent by providing a ready source of cations to exchange with and, thereby, enable the release of, the antimicrobial metal ions. Similarly, the antimicrobial agent may be used in conjunction with or contain a discoloration inhibitor, preferably inorganic discoloration inhibitors such ammonium compounds. Especially preferred are ion-exchanged ammonium ions which are found to improve color stability, i.e., reduce the manifestation of discoloration due to, for example, the interaction of silver ions with other ions or compounds present in the binder or coating composition. Such additional components and/or ions are desirable so long as they are biocompatible and do not interfere with the bioefficacy of the antimicrobial agent. Nevertheless, given the fact that the antimicrobial activated carbon materials are not concerned with color and the possibility exists that the activated carbon may adsorb certain of these other additives, it may be prudent to limit their use of the use of certain such additives to those instances where their benefit is needed and outweighs any adverse impact on the efficacy or life of the purification properties of the activated carbon itself.

As noted above, the preferred antimicrobial agents are those wherein the ion-exchange material or carrier is a zeolite. Antimicrobial zeolites typically comprise from about 0.1 to about 25 wt %, preferably from about 0.3 to about 20 wt %, most preferably from about 2 to about 10 wt %, of the antimicrobial metal ion or ions based upon 100% total weight of antimicrobial zeolite. In addition, the antimicrobial zeolites may also contain ion-exchanged ammonium ions, which may be present at a level of up to about 20 wt %, based on the total weight of the antimicrobial zeolite. Preferably, however, it is desirable to limit the content of ammonium ions to from about 0.1 to about 2.5 wt % of the zeolite, more preferably from about 0.25 to about 2.0 wt %, and most preferably, from 0.5 to about 1.5 wt %.

The zeolites to be used in the practice of the present invention may be either natural zeolites or synthetic zeolites. “Zeolites” are aluminosilicates having a three dimensional skeletal structure that is represented by the formula: XM_{2/n}O—Al₂O₃—YSiO₂—ZH₂O, wherein M represents an ion-exchangeable ion, generally a monovalent or divalent metal ion; n represents the atomic valency of the metal ion; X and Y represent coefficients of metal oxide and silica, respectively; and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. The present invention is not restricted to use of these specific zeolites.

The ion-exchange capacities of these zeolites are as follows: A-type zeolite=7 meq/g; X-type zeolite=6.4 meq/g; Y-type zeolite=5 meq/g; T-type zeolite=3.4 meq/g; sodalite=11.5 meq/g; mordenite=2.6 meq/g; analcite=5 meq/g; clinoptilolite=2.6 meq/g; chabazite=5 meq/g; and erionite=3.8 meq/g. These ion-exchange capacities are sufficient for the zeolites to undergo ion-exchange with ammonium and antimicrobial metal ions.

The specific surface area of preferred zeolite particles is preferably at least 150 m²/g (anhydrous zeolite as standard) and the SiO₂/Al₂O₃ mole ratio in the zeolite composition is preferably less than 14 and more preferably less than 11.

The antimicrobial metal ions used in the antimicrobial zeolites are retained in and on the zeolite particles through an ion-exchange reaction. Antimicrobial zeolites in which the antimicrobial metal ions are solely or predominately adsorbed or attached without an ion-exchange reaction typically exhibit an overall decreased bactericidal effect and their antimicrobial effect is not long lasting. Nevertheless, it can be advantageous for imparting quick antimicrobial action to maintain a sufficient amount of surface adsorbed metal ion in addition to the ion-exchanged metal ion.

A preferred antimicrobial zeolite for use in the invention is type A zeolite containing ion-exchanged silver, zinc, and/or copper ions in combination with ammonium ions; more preferably combinations of the silver and copper ions with the ammonium ions or just silver ions and ammonium ions. A number of antimicrobial zeolites suitable for use in the practice of thee present invention are distributed by AglON Technologies, Inc., of Wakefield, Mass., USA, under AglON trademark. One grade, AW10D, contains 0.6% by weight of silver ion-exchanged in Type A zeolite particles having a mean average diameter of about 3μ. Two additional grades, AG10N and LG10N, each contain about 2.5% by weight of silver ion-exchanged in Type A zeolite particles having a mean average diameter of about 3μ and 10μ, respectively. Another grade, AJ10D contains about 2.5% silver, about 14% by weight zinc, and between about 0.5% and 2.5% by weight ammonium ion-exchanged therein in Type A zeolite having a mean average diameter of about 3 p. Another grade, AK10D, contains about 5.0% by weight of silver ion-exchanged in Type A zeolite particles having a mean average diameter of about 3μ. Finally, another grade, AC10D, consists of about 6.0% by weight of copper and about 3.5% by weight silver ion-exchanged in Type A zeolite particles having a mean average diameter of about 3μ. Though all of the foregoing are suitable for use in the practice of the present invention, depending upon the specific application, the desired longevity, etc., it is anticipated that a preferred embodiment may employ somewhat larger particle size antimicrobial zeolites, e.g. 10μ, with a high silver ion loading, e.g., 5%, with or without zinc ions, e.g., about 14% zinc.

The antimicrobial agent to be used in the practice of the present invention can be used in its neat form or it may be encapsulated as described in United States Published Patent Application No. US2003-0118664 A1 (U.S. Ser. No. 10/032,372 filed Dec. 21, 2001 by Trogolo et al.), which is incorporated herein by reference. Generally speaking, the encapsulated antimicrobial agent is in the form of microcapsules or particles that comprise either a single particle or, most preferably, a plurality (several to several hundred or more) of particles of the antimicrobial agent encapsulated within a hydrophilic polymer. The encapsulated antimicrobial agent may be of many shapes and may deform somewhat during processing of the coating. Generally, the encapsulated antimicrobial agent will be in the form of particles having a low aspect ratio, for example, on the order of from 1 to about 4, preferably from 1 to about 2, most preferably from 1 to about 1.5. However, it is also contemplated that microcapsules may be of a high aspect ratio as taught in United States Published Patent Application No. US2003-0118658 A1 (U.S. Ser. No. 10/032,370 filed Dec. 21, 2001 by Trogolo et al), also incorporated herein by reference. These high aspect ratio microcapsules are typically in the shape of flakes and fibers whose aspect ratio is up to 100 or more, but typically is less than about 30.

The hydrophilic polymers suitable for use in encapsulating the antimicrobial agent are those that can absorb sufficient water to enable the encapsulated particle to exhibit good antimicrobial behavior, i.e., to allow for the migration and release of the antimicrobial active agent. These polymers are characterized as having water absorption at equilibrium of at least about 2% by weight, preferably at least about 5% by weight, more preferably at least about 20% by weight, as measured by ASTM D570. Especially suitable hydrophilic polymers include those having water contents at equilibrium of from about 50 and to about 150% by weight.

The encapsulating hydrophilic polymers, hereinafter oftentimes referred to as the encapsulant, are typically comprised of substantial quantities of monomers having polar groups associated with them, such that the overall polymeric composition is rendered hydrophilic. The polar groups can be incorporated into the polymer main chain as in for example polyesters, polyurethanes, polyethers or polyamides. Optionally the polar groups can be pendant to the main chain as in for example, polyvinyl alcohol, polyacrylic acids or as in ionomers such as Surlyn®. Surlyn® is available from Dupont and is the random copolymer poly(ethylene-co-methacrylic acid) wherein some or all of the methacrylic acid units are neutralized with a suitable cation, commonly Na⁺ or Zn⁺². While not being limited by way of theory, it is believed that the inclusion of polar groups allows water to more readily permeate the polymer and consequently, to allow slow transport of the metal ion through the encapsulating polymer layer. Such encapsulants may be thermoplastic or they may be thermoset or cross-linked.

A number of specific hydrophilic polymers suitable for use as the encapsulant include, for example, (poly)hydroxyethyl methacrylate, (poly)hydroxypropyl methacrylate, (poly)glycerol methacrylate, copolymers of hydroxyethyl methacrylate and/or methacrylic acid, polyacrylamide, hyaluronan, polysaccharides, polylactic acid, copolymers of lactic acid, (poly)vinyl pyrrolidone, polyamides such as Nylon 6,6, Nylon 4,6 and Nylon 6,12, cellulosics, polyureas, polyurethanes and certain polyesters containing a high percentage (at least about 10% by weight, preferably at least about 25% by weight or more) of polyalkylene oxide.

The hydrophilic polymer may be a copolymer containing at least a substantial amount of at least one or more of the above-mentioned hydrophilic monomers, including, for example, styrene/methacrylic acid/hydroxyethyl methacrylate copolymers, styrene/methacrylic acid/hydroxypropyl methacrylate copolymers, methylmethacrylate/methacrylic acid copolymers, ethyl methacrylate/styrene/methacrylic acid copolymers and ethyl methacrylate/methyl methacrylate/styrene/methacrylic acid copolymers, copolymers based upon the cellulosics, and copolymers which utilize vinylpyrrolidone monomers, among numerous others, especially copolymers of n-vinylpyrrolidone and polymethylmethacrylate.

Other encapsulants include polyvinyl acetate, polyvinyl alcohol, and copolymers of polyvinyl alcohol and polyvinylacetate, polyvinylchloride, copolymers of polyvinylacetate and polyvinylchloride and hydroxyl-modified vinyl chloride/vinyl acetate copolymers.

Polyurethanes containing a high percentage (at least about 10% by weight, preferably at least about 25% by weight or more) of polyalkylene oxide are especially useful in this invention.

Preferably the encapsulating hydrophilic polymer is chosen from polyhydroxyethyl methacrylate, polyacrylamide, polyvinylpyrrolidinone, polyurea, polysaccharides, polylactic acid, poly(meth) acrylic acid, polyurethane and copolymers thereof. More preferably, the hydrophilic polymer is hydrophilic polyurethane, such as the TECOPHILIC® polyurethane sold by Thermedics of Woburn, Mass. or a lightly cross-linked polymer based on n-vinylpyrrolidone and methylmethacrylate sold under the trade designation AEP Polymers by I H Polymeric Products Limited of Kent, England.

While the encapsulated antimicrobial agent may be in the form of individually encapsulated antimicrobial particles having a coating thickness of up to 15μ, more typically and preferably, they are in the form of larger microcapsules containing multiple antimicrobial particles, especially of the ion-exchange type. The latter typically comprise from about 5 wt % to about 65 wt %, preferably from about 20 wt % to about 50 wt % of the antimicrobial agent based on the total weight of the encapsulated antimicrobial agent. Although the latter microcapsules may have a mean average diameter of up to and over 2000μ, for use in the present invention their size will be much smaller, generally they will have a mean average diameter of up to about 300μ, preferably from about 30μ to about 200μ, most preferably from about 50μ to about 150μ. Of course smaller or larger microcapsules can be used depending upon the size of the activated carbon particles.

Encapsulated antimicrobial agents are especially useful where the binder is not a hydrophilic material. This is because the transport mechanism by which the ion-exchange type antimicrobial agents work is reliant upon a liquid medium, preferably water, bringing ions to the antimicrobial particle to exchange with and thereby release the antimicrobial metal ions. Thus, unless there are pathways through the binder or the binder is a hydrophilic material, at least a portion of the surface of the ion-exchange type antimicrobial agent must be exposed in order for a given particle of the antimicrobial agent to be effective. With the former, such pathways may be naturally occurring, well defined pathways or channels as in the case of porous polymers and inorganic binders or they may be in the form of molecular sized pores that exist between molecules and/or polymer chains in the case of thin layers of the binder, i.e., nano- or angstrom scale.

The encapsulated antimicrobial agents enhance bioefficacy on two fronts. First, the significantly larger particle size of the encapsulated antimicrobial agents increases the likelihood that any one particle will have an exposed surface or touch or in be close proximity to the surface of the binder. Secondly, because the entire amount of the antimicrobial active within a given particle of the encapsulated antimicrobial agent is accessible, those particles having a plurality of particles of the antimicrobial agent incorporated therein serve as large reservoirs of the antimicrobial active, i.e., the antimicrobial metal ions. Furthermore, because the degree of hydrophilicity controls, in part, the release rate of the antimicrobial metal ions, these materials also provide for greater longevity combined with the excellent release. And, where the binder is itself a hydrophilic polymer, the use of the encapsulated antimicrobial agent allows one to further regulate the release of the antimicrobial agent by encapsulating the antimicrobial agent with a hydrophilic polymer having a different degree of hydrophilicity. For example, if the encapsulating material is of a lower hydrophilicity than the binder, it will serve to slow the release of the antimicrobial ions.

Binder materials suitable for use in the practice of the present invention include hydrophilic polymers, thermoset resins, thermoplastic polymers and inorganic binders such as silicates. As applied to the activated carbon particles, the binder is in the form of a ‘curable’ coating composition. These coating compositions may be single part or multi-part compositions. Typical single part compositions are flowable 100% solids curable compositions; “solvent” based, especially water-based, systems such as true solutions, dispersions or colloids; and curable liquid compositions. Generally, these compositions cure or set upon exposure to the atmosphere or other curing conditions. Alternatively, the single part coating composition may be in the form of fine and ultra-fine particle size powder coatings. Multipart compositions typically comprise two or more parts liquid curable/reactive components that are essentially shelf stable as long as the two parts remain isolated from one another but cure or become curable upon mixing of the two or more parts.

Although the antimicrobial coating may be applied to the whole of the exposed surface of the activated carbon, it is preferred that no more than 60%, more preferably no more than 50%, most preferably no more than 40%, of the exposed surface area have the antimicrobial coating applied thereto. Where more than 60% of the surface is covered with the coating, the coating will preferably, and, in the case of more than 80% coverage, must be of such a viscosity and/or posses such surface tension characteristics that the coating will not flow into or block the pores of the activated carbon, at least not to any substantial extent. Such characteristics may be inherent to the coating composition or they may be imparted to the same by the addition of viscosity modifiers and surface active agents, especially surfactants, respectively. Since it is impossible to ensure that no blockage occurs, the present invention will tolerate some blockage of the pores provided that the water flow-through of a filter element made from the antimicrobial activated carbon is not significantly impacted. Specifically, besides adversely impacting purification capabilities, too much pore blockage will result in marked reduction in the flow-through rate so as to render the filter impractical for its intended use. Though not intending to be bound by theory, it is believed that the present invention can tolerate blockage whereby the percent pressure drop occasioned by the application of the coating in a water filter prepared from said antimicrobial activated carbon particles will be no more than about 150%, preferably no more than about 130%, most preferably no more than about 120% of the pressure drop across the water filter media seen with an identical filter made with the same, but untreated, activated carbon particles. So long as the pores are not blocked, the percent coverage of the exposed surface area of the activated carbon is not so critical since the greatest portion of the overall surface area of the activated carbon particles, thus the adsorptive capabilities, is represented by the unexposed surface of the channels and tunnels in and through the activated carbon.

The chemistry or formulation of the thermoset or thermoplastic coating compositions vary widely and are selected based on the desired physical properties of the coating compositions, the mode of application (e.g., solution based, curable 100% solids or powder coating), the pot life (if applicable), the cure mechanism (i.e., heat, UV light, moisture, etc.), and the environmental conditions to which they are exposed in use. Typically, in the case of thermoset coatings the choice of polymer or polymerizable components is based on the cure method and pot life as well as the adhesion, wear, and appearance characteristics or properties. In the case of thermoplastic coatings, selection of the thermoplastic polymer is based on the solvent needed and/or the ease of application, especially as powder coatings, as well as their adhesion, wear and appearance characteristics or properties. Suitable thermoplastic polymers include, but are not limited to, polypropylene, polyethylene, polystyrene, ABS, SAN, polybutylene terephthalate, polyethylene terephthalate, nylon 6, nylon 6,6, nylon 4,6, nylon 12, polyvinylchloride, polyurethanes, silicone polymers, polycarbonates, polyphenylene ethers, polyamides, polyethylene vinyl acetate, polyethylene ethyl acrylate, polylactic acid, polysaccharides, polytetrafluoroethylene, polyimides, polysulfones, and a variety of other thermoplastic polymers and copolymers. Suitable thermoset or cross-linkable coatings include, but are not limited to, phenolic resins, urea resins, epoxy resins, including epoxy-novolak resins, polyesters, epoxy polyesters, acrylics, acrylic and methacrylic esters, polyurethanes, acrylic or urethane fortified waxes and a variety of other thermoset or thermosettable polymers and copolymers. Thermoset coating systems based on epoxy resins, whether 100% solids or aqueous dispersions/latexes, are especially preferred due to their excellent adhesive properties and durability. Suitable epoxy resin systems include those sold by Corro-Shield of Rosemont, Ill. as well as Burke Industrial Coatings of Vancouver, Wash..

Inorganic binder systems are also suitable for use in the practice of the present invention. Exemplary of such binder systems are the silicates, especially sodium silicate sold by PQ Corporation of Berwyn, Pa.

The preferred binders for use in the practice of the present invention are the hydrophilic polymers. These may be either thermoset (i.e., curable thermosetting or cross-linking polymer compositions) or thermoplastic compositions. Suitable hydrophilic polymers include those previously mentioned and discussed at length above with respect to the encapsulation of the antimicrobial agent. Especially preferred hydrophilic binders are those based poly(meth)acrylates and poly (meth)acrylic acids or on cross-linked polyurethanes described in, e.g., U.S. Pat. No. 6,238,799 and U.S. Pat. No. 6,866,936 and available from Surface Solutions Laboratories of Carlisle, Mass. Those skilled in the art are well versed in and will readily recognize how to prepare coating compositions comprising such hydrophilic materials.

In preparing the antimicrobial activated carbon of the present invention, the antimicrobial agent may be directly incorporated into the liquid or flowable 100% solids binder coating composition or into the powder coating material priorto its application to the activated carbon particles or, in the case of the liquid or flowable 100% solids binder systems, it may be applied subsequent to the application of these binder systems. In the latter instance, the antimicrobial agent is either dusted or sprinkled over the wetted activated carbon particles prior to cure of the binder coating or it may be applied in a mist of an activator or activator solution which effectuates cure (including evaporation of a solvent, if applicable) of the binder coating.

Generally speaking, the amount of antimicrobial agent incorporated into or employed in conjunction with the binder is typically from about 10 wt % to about 50 wt %, preferably from about 20 wt % to about 30 wt %, based on the total weight of the cured antimicrobial binder. In the case of encapsulated antimicrobial agents, the wt% of the antimicrobial agent is based on the amount of antimicrobial agent itself (including the carrier), exclusive of the encapsulant.

Where the antimicrobial additive is incorporated into the coating composition prior to application, the rate of application of the binder coating composition should be kept low in order to maintain as thin a coating as possible, especially for non-hydrophilic binders. This is especially of concern where the binder material has a tendency to form a skin over the particles of the antimicrobial agent or is of sufficiently low viscosity that the particles of the antimicrobial agent sink: particularly if the non-hydrophilic binder does not contain pores or other pathways which allow transport of the ions through the binder matrix. The rate of application is less of concern where the antimicrobial agent is applied to the wetted surface of the activated carbon particle since essentially all of the antimicrobial agent will be exposed at the coating surface. However, here again concern should be made to avoid non-hydrophilic coatings that are too thick and allow the particles of the antimicrobial agent to sink to a depth that the particles are completely covered by the binder material.

Preferably, the binder composition is a hydrophilic composition, as defined above. The use of such materials avoids the aforementioned concerns with respect to coating thickness and lack of exposure of the antimicrobial agent to the coating surface. Furthermore, hydrophilic coatings allow for the application of multiple layers of the antimicrobial coating to the activated carbon particles. This buildup of coating allows one to increase the amount of antimicrobial agent. Additionally, and perhaps more importantly, it also enables one to design the coating such that each successive layer contains less and less antimicrobial agent with perhaps none in the outermost surface layer. In accordance with Flicks Law, the use of multiple layers of successively less concentration of antimicrobial agent allows one to achieve an essentially constant release rate for the antimicrobial agent until all or substantially all of the antimicrobial agent is spent. Thus, by proper selection of the concentration of each layer as well as the hydrophilicity of the hydrophilic binder, one can essentially predetermine the level of release as well as the bioefficaceous life of a filter made with those materials.

Though the particle size of the activated carbon and the viscosity and surface tension characteristics of the uncured binder matrix will influence the thickness of the antimicrobial coating to be applied, typically in the case of granular activated carbon used in consumer water filters, the thickness of the coating will be less than about 50 p, preferably from about 1μ to about 30μ, most preferably from about 1μ to about 20μ. Somewhat thicker coatings may be found with multi-layered coatings; however, it is preferred to keep within the aforementioned ranges. In essence, unless the amount of exposed surface to be covered is low, the thickness of the antimicrobial coating should kept low in order to minimize any blockage of the pores in the region of coverage. As known to those skilled in the art, the tendency or likelihood for a coating to pull away from a pore opening decreases with higher viscosity binder compositions and as the thickness of the coating applied increases.

Notwithstanding the foregoing, when the antimicrobial agent is applied to the activated carbon in the form of an antimicrobial thermoplastic powder, the coating thickness tends to be higher, oftentimes up to 200μ or so. Preferably, though, the thermoplastic coating is kept as thin as possible and practical, e.g., between about 20μ and 150μ. Of course the ultimate thickness of the thermoplastic coating will depend upon the proper melt application, the melt characteristics of the thermoplastic polymer, especially its melt viscosity, and the size of the antimicrobial powder coating particles themselves. As technology improves to enable the formation of smaller and smaller particle size antimicrobial powders, it is anticipated that coatings in the lower end of the aforementioned thickness range and thinner may be possible. Furthermore, the amount of the antimicrobial thermoplastic powder should be kept relatively low, generally no more than about 30% by weight, preferably no more than about 20% by weight, most preferably no more than about 15% by weight, based on the weight of the GAC in order to avoid excessive coating and agglomeration of the GAC, especially where the GAC is to be employed in a free powder or loose fill state.

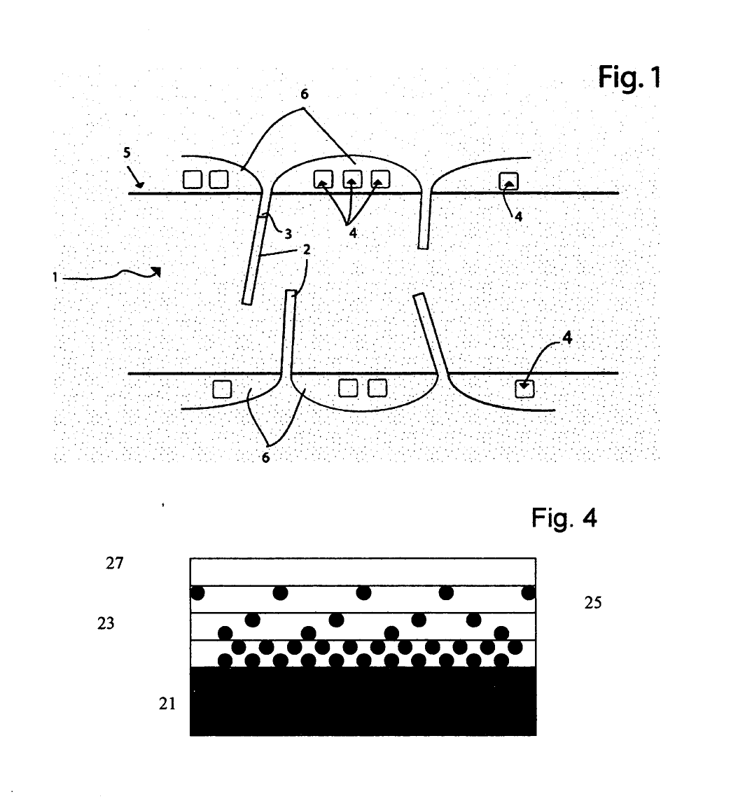

For ease of understanding, FIG. 1 presents a cross-sectional schematic of a portion of an antimicrobial activated carbon particle according to the present invention. As shown, the activated carbon particle (1) has a plurality of internal pores (2) of varying pore diameter (3). Though depicted as being almost well like in structure, the pores actually branch out into a series of channels or tunnels, many of which interconnect and form passageways through the activated carbon particles. As shown, the antimicrobial agent (4) is bonded to the exposed surface (5) of the activated carbon particles (1) by means of the binder or coating composition (6).

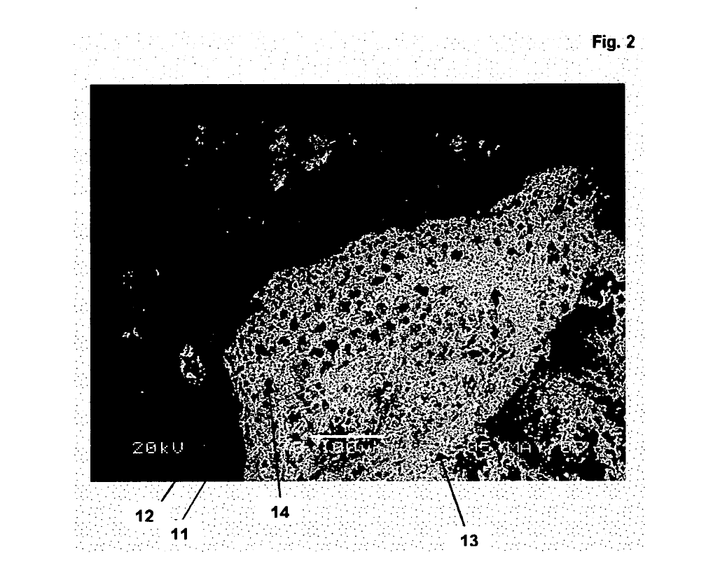

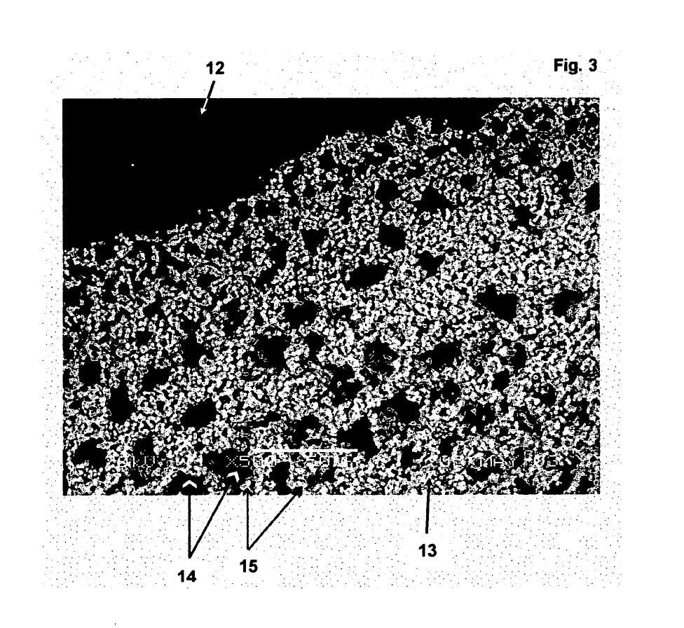

FIGS. 2 and 3 are scanning electron microscope photographs of a portion of an actual antimicrobial granular activated carbon particle made in accordance with the practice of the present invention. Specifically, FIG. 2 shows an activated carbon particle (11) having uncoated surface (12) and coated surface (13). Due to the low surface tension of the coating, the pores (4) of the activated carbon particle are not blocked or plugged with the coating. To more clearly show this effect, FIG. 3 is a close up view of a portion of the particle in FIG. 2 wherein the pores (14) in the coated surface (13) are clearly visible. Also visible in this photograph are the individual particles of the antimicrobial agent, a silver zeolite, which appear as white snowballs on the activated carbon particle surface (15).

FIG. 4 presents a cross-sectional view of a portion of an activated carbon particle (21) to which multiple layers of antimicrobial coatings (23) have been applied. As depicted, each successive layer has less and less antimicrobial agent (25). Additionally, this figure shows and outermost layer (27) of a binder material that is free of antimicrobial agent. This may serve as a protective layer and/or to help control or regulate the release rate for the antimicrobial agent.

The antimicrobial activated carbon may be prepared by a number of different methods depending upon the binder composition and its method of cure, if applicable. Typically the antimicrobial agent is applied as a component of a liquid curable coating or binder system or is applied topically to the wetted surface of the activated carbon wherein the surface is wetted with a liquid curable coating or binder system. In one embodiment, the coated activated carbon is prepared by spreading a single layer of activated carbon particles, especially granular activated carbon (GAC), onto a flat surface and spraying a thin layer of the antimicrobial containing coating onto the particles. In this method, the coating is applied to only one side of the GAC, leaving the remaining surface of the GAC free of coating and unchanged as to the absorptive properties. Of course, if one does not want full coverage, one could also have the spray intermittent or sputter so that not all of the exposed upper surface of the activated carbon particles is coated. On a production scale this process can be performed using a conveyor belt or a rotating table or disc which carries essentially a monolayer of the activated carbon particles under one or more spray nozzles that apply the antimicrobial coating or binder to the exposed surface of the activated particle either as a continuous or discontinuous film or coating. In the case of the rotating disc, the apparatus will include a dam or barrier that directs the treated antimicrobial off the rotating disc. Additionally, if a multi-layer coating is desired, the conveyor or disc can move the material past a plurality or series of nozzles, ultimately building a multi-layer coating. Similarly, if the coating or binder system is a multi-part system, there will be a plurality of spray nozzles, with one applying one part and a second, preferably downstream of the first, applying the second part whereby cure is initiated upon contact of the two parts. Alternatively, where the speed of cure is not instantaneous or nearly so, it is possible for the two parts to be sprayed simultaneously so that good and intimate mixing of the two parts occurs.

As noted above, cure of the binder system may be effectuated by a chemical reaction or it may simply be a matter of driving off the solvent in which the binder system is soluble/dispersed, typically water. Depending upon the cure mechanism of the coating or binder system, the coating apparatus may further include or have associated therewith one or more radiant heaters, UV lamps, or the like along the conveyor belt or at another location along the path of the rotating disc or table which effectuates or speeds up cure of the binder material, e.g., heat will speed up the evaporation of the solvent. In the case of multi-layered coatings, a heater, UV lamp, etc. may be intermediate each coating application station to enable the first layer to cure before application of the second layer.

As mentioned previously, the antimicrobial agent may be present as a constituent of the curable coating or binder composition or, as appropriate, of one or both components of a multipart curable coating or binder system. Alternatively, the antimicrobial agent may be applied to the activated carbon particles after the coating or binder compositions has been applied but before its cure, i.e., while the surface is still wet or tacky. Here, the antimicrobial agent may be sifted or dusted onto the wetted surface. The coated activated carbon particles are then subjected to curing conditions, e.g., UV light, heat, etc. as necessary, to effectuate full cure of the binder system upon which the antimicrobial particles are bound. In this process, because of the expense of most antimicrobial agents of the type employed in the present invention, it will also be preferable to have a means to recover that antimicrobial agent which does not adhere to the wetted surface. For example, in the dusting station, the conveyor belt or surface upon which the activated carbon particles are being carried may be an open mesh or small grates that allows the antimicrobial agent that does not stick to the wetted particles to pass through and be collected.

In yet another alternative method of making the antimicrobial activated carbon particles, the apparatus mentioned above may further include a means by which the particles are flipped and sent through the coating apparatus again or through a second coating apparatus to apply the antimicrobial coating to the other side of the activate carbon particles. Such means may simply be cascading conveyors where the one drops the treated activated carbon particles onto another or the particles may be collected in a continuous or batch-wise manner between two surfaces that essentially immobilize the particles so they can be flipped. Other methods for accomplishing the same result will be readily recognized by those skilled in the art. Furthermore, it is appreciated that the certain methods will be more efficient than others in flipping the activated carbon particles and that, due to their varied shape, some particles will rotate to some extent on their own; however, the gist of the process of the present invention is still realized where a substantial portion of the particles are covered on both “sides”.

Yet another method of applying the antimicrobial coating to the activated carbon particles, especially where one is not so concerned with percent or consistent coverage from one particle to another or where one wants substantially complete coverage, is to employ a vessel with a mixing arm or blade as well as one or more spray nozzles overhead whereby the antimicrobial curable coating is applied to the activated carbon particles as they are being continuously churned by the mixing arm or blade. Alternatively, the same process may be achieved in a rotating drum where the drum is on its side and the spray nozzle applies the coating as the drum rotates, thereby churning the activated carbon particles. Both of these processes can be employed as batch processes where a given amount of activated carbon particles is treated with a given amount of the antimicrobial curable coating composition or as continuous processes where the coating is continuously applied as activated carbon particles are continuously added and withdrawn from the mixing vessels. Both processes, depending upon the residence time in the vessel, enable one to produce antimicrobial activated carbon particles wherein 100% or nearly so of the exposed outer surface of the particle is covered with the antimicrobial binder composition. In the batch processes mixing may continue until the cure is effected; whereas, in the continuous processes treated activated carbon particles are cured as they flow out of or following their exit from the mixer vessel. For example, where the binder is a UV curable composition, the exit chute or conveyance means may have a UV light associated with it that shines upon the wetted particles.

In yet another embodiment, the antimicrobial agent is incorporated into a thermoplastic binder and the mixture converted to a fine, preferably an ultra-fine, powder. These powder coatings are preferably of a particle size that is on low end or smaller than those of traditional, commercial powder coating compositions. Most preferably, the powder coating particles will be of a size that is comparable to, but preferably less than, the particle size of the activated carbon itself. Especially preferred are antimicrobial thermoplastic powders having a mean particle size of less than about 500μ, preferably less than about 300μ, most preferably less than about 200μ. As technology improves, particles of even smaller particles size will be suitable, and preferred. Indeed, it would be especially desirable to have antimicrobial thermoplastic particles of less than about 100μ, preferably from about 20μ to about 50μ, most preferably about 30μ.

In preparing the antimicrobial activated carbon using these antimicrobial thermoplastic powders, the antimicrobial powder is dry blended with the activated carbon particles under increasing elevated temperatures until the thermoplastic binder melts and adheres to the activated carbon particles. Alternatively, especially if there is concern with or experience with clumping of the thermoplastic powder particles, the activated carbon particles may be heated to a temperature near or, preferably at or above, the melt temperature of the thermoplastic binder and the antimicrobial thermoplastic binder particles gradually added to the heated activated carbon particles with mixing so that the powdered antimicrobial thermoplastic particles collide with the heated activated carbon particles and adhere to the same as a result of a melt fusion at the interface between the activated carbon particle and the antimicrobial thermoplastic particle. In yet another alternative, the antimicrobial powder coating material may be combined with the activated carbon as it is cooling down following the high temperature activation treatment. In each of these instances, it is preferred that the thermoplastic be selected to have a low melt viscosity such that the polymer melt will wick across the surface of the activated carbon. Surface coating with the antimicrobial thermoplastic is also aided by the mixing process which tends to smear the thermoplastic on the surface of the activated carbon.

The antimicrobial activated carbon materials made in accordance with the present invention may be used in most any application where conventional activated carbons are employed. They are especially suited for use in liquid and gas purification and filtration applications, most especially in water purification and filtering applications. These materials are particularly suited for removal of bacteria and other contaminants from water used in the biotechnological field, especially where certain microorganisms are to be propagated, as well as in the pharmaceutical, specialty chemical (especially for food and pharmaceutical grade products), food and beverage processing, and medical service industries. Such purification and decontamination is not only important for water that is incorporated into the end products of such industries, but is also vital for water used in processing, cleaning operations, and the like as well as prevention of putrification of stored water. Furthermore, because of their controlled ion release characteristics, especially as compared to conventional silverized activate carbon and other quick release antimicrobial systems, they are particularly suited for use in consumer/potable water filtration and purification applications, including, for example, in filter elements for in-line filtration and purification such as under sink filters and tap/faucet attachment type filters. They are also especially suitable for use in stand alone water filtration and dispensers systems, including those dispensing bottled or tap water, and, in particular, portable, water dispensers that purify tap water such as the water carafes. Finally, these filter media and associated filter elements are well suited for use in the burgeoning industry of bottled water.

The antimicrobial activated carbon materials according to the present invention are suitable for use in the manufacture of many types of filter elements including loose fill filters and sintered filters. They are especially beneficial in loose fill filters since attempts at incorporating particulate antimicrobial agents in loose fill activated carbon filters have been plagued with a number of problems including the settling of the antimicrobial agent in the chamber containing the loose fill and, worse, the actual passing of small particles of the antimicrobial agent through the filters themselves. Though, as noted in the background section, others have bound the antimicrobial agent to large particles of a filler or polymer material, the use of such large particles supplants activated carbon particles thereby reducing the overall life expectancy of the filter element. By directly applying the antimicrobial agent in a thin coating to the surface of the activated carbon particles, the impact on the amount of activated carbon that may be placed in a given volume is insubstantial.

As known in the art, sintered activated carbon filters are prepared by uniformly dry blending activated carbon with thermoplastic particles and then compression molding the same at elevated temperatures to enable the thermoplastic particles to melt fuse to one another as well as the activated carbon particles. This process allows for the formation of a uni-body filter, avoiding concern with loose materials, having tortuous pathways that trap small artifacts and organisms. Depending upon the type and chemistry of the binder material employed to bind the antimicrobial agent to the activated carbon particles, the binder may participate in the sintering process, enabling one to use less thermoplastic particles than one might otherwise. Furthermore, since most thermoplastics employed in the sintering process are not hydrophilic, the use of a hydrophilic binder will offset, to some extent, the adverse impact on the performance and life of the activated carbon resulting from the blockage of the pores where the thermoplastic particles fuse to the activated carbon particles. Specifically, if a thermoplastic particle fuses to an area of the activated carbon particle where a hydrophilic polymer binder is present, without completely encapsulating the hydrophilic binder, the exposed area of the hydrophilic binder, no matter how small, still represents a pathway by which the antimicrobial agent can be released from the coating and provide antimicrobial efficacy. A happenstance not possible with silverized activated carbon.

Generally speaking, filter elements, whether loose fill or sintered, can be made by any of the known methods for making such filters using traditional activated carbon. Similarly, the antimicrobial activated carbon particles of the present invention may be incorporated into any filter media and filter media compositions where traditional activated carbon particles, including silverized activated carbon particles, are employed. Such compositions may include any number of additional components including ion exchange resins, chelating agents, inorganic media which adsorb such materials as perchlorates, nitrates, calcium or heavy metals such as lead, mercury, arsenic, chromium, etc. Since such materials and methods are well known in the art, they are not described further.

The following examples are presented in order to aid in the full understanding of the present invention and demonstrate its advantages over the current commercial technology. These examples are merely illustrative of the invention and are not to be deemed limiting thereof. Those skilled in the art will recognize many variations that are within the spirit of the invention and scope of the claims.

### EXAMPLES 1 and 2

### COMPARATIVE EXAMPLE 1

An antimicrobial curable coating composition was prepared by adding AglON AJ10D silver zeolite antimicrobial agent (2.5% silver) (AglON Technologies, Inc., Wakefield, Mass.) to a hydrophilic acrylic binder/coating supplied by Surface Solutions Laboratories of Concord, Mass. in an amount sufficient to elevate the level of the AJ10D antimicrobial agent to 58% by weight, based on the total solids of the antimicrobial curable coating composition. The antimicrobial coating composition was then applied to conventional consumer water filter grade granular activated carbon by two different methods. In Example 1, the activated carbon was placed in a vessel and sprayed with the coating as the particles were churned in order to coat essentially 100% of the exposed surface. In Example 2, the activated carbon particles were laid out in a monolayer on a surface and a layer of the antimicrobial coating sprayed over the particles to essentially cover the upper, exposed surface. The coated activated carbon particles were then placed in separate gravity flow filter cartridges of the size and shape of a commercial Brita carafe filter. The amount of the antimicrobial granular activated carbon added to each filter cartridge was the same as typically employed in commercial filters, such as the Brita filter. These two cartridges and a commercial Brita consumer carafe filter were then evaluated in the following experiments.

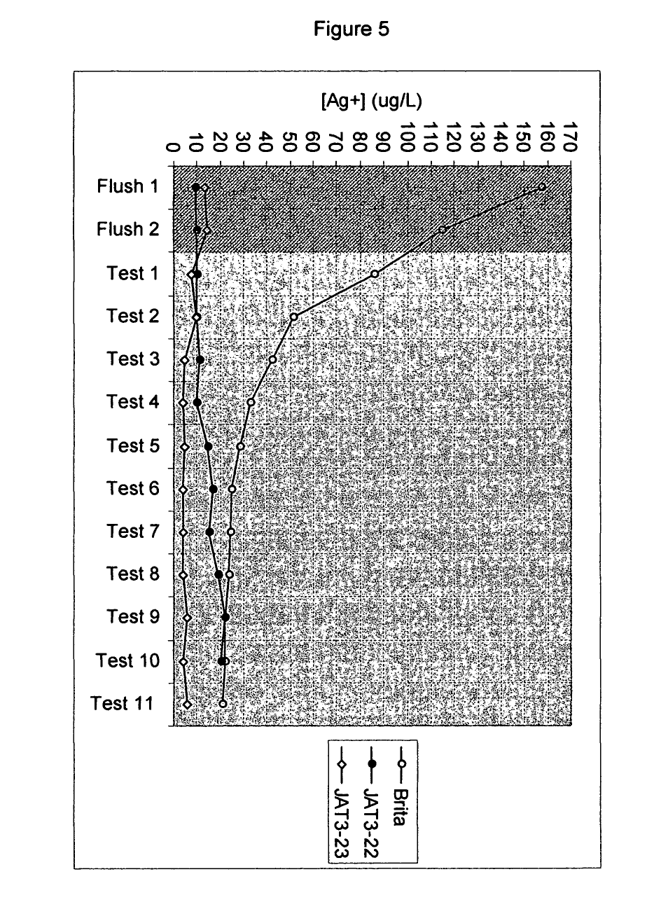

Each of the filters was placed in the consumer carafe for which the filter was made and one (1) liter of tap water (MWRA Wakefield, Mass.) was added to fill the filling reservoir. Each carafe was allowed to stand at room temperature until the full liter of water had passed through the filter element. The effluent was then filtered using a 0.25μ syringe filter to remove any antimicrobial or activated carbon particles. The silver concentration in the effluent waster was then measured using a graphite furnace atomic absorption spectrometer. This process was repeated for each carafe until each carafe had filtered thirteen (13) liters. The results, presented as silver [Ag+] in micrograms per liter [μg/l] water are presented in Table 1 and in FIG. 5.

As seen in Table 1 and FIG. 5, the commercial Brita filter, Comparative Example 1, released massive amounts of silver during the first several uses as well as comparatively high levels of silver for the following uses. Indeed, as seen from the results in Table 1, the first two (2) liters of treated water exceeded US EPA Safe Drinking Water standards while the first four (4) liters of treated water exceeded the recommended standard of the World Health Organization. None of the water treated using the filters of the present invention, Examples, 1 and 2, exceeded or even came close to reaching the lower standard of the World Heath Organization.

Even if one were to assume the consumer discarded the first two liters of the commercial filter treated water, a single person consuming the next ten (10) liters of treated water would have consumed a total of 356.65 micrograms of silver as compared to just 166.32 micrograms and 86.54 milligrams of silver for the first twelve (12) liters of treated water using the filters of Examples 1 and 2, respectively. Though the level of silver release in the commercial filter does eventually fall to levels consistent with the filters of the present invention, given the limited life of these filters and the need for constant replacement, one can only imagine the huge difference in silver consumed by an individual over a period of months, years and, especially, a lifetime.

TABLE I

| | Sample 1 | Sample 2 | Sample 3 |
|---|---|---|---|
| Flush 1 | 157.2 | 8.975 | 13.02 |
| Flush 2 | 114.8 | 9.629 | 14.65 |
| Test 1 | 86.05 | 9.65 | 7.567 |
| Test 2 | 51.47 | 10.07 | 9.968 |
| Test 3 | 42.27 | 10.76 | 4.6 |
| Test 4 | 32.67 | 9.713 | 4.3 |
| Test 5 | 28.13 | 14.2 | 4.468 |
| Test 6 | 24.92 | 16.97 | 4.107 |
| Test 7 | 23.95 | 15.23 | 4.276 |
| Test 8 | 23.56 | 18.76 | 4.089 |
| Test 9 | 21.71 | 21.98 | 5.86 |
| Test 10 | 21.92 | 20.44 | 4.197 |
| Test 11 | 20.92 | | 5.5 |

Of further significance is the impact this massive and consistently high release has on the efficacious life of the filter element itself. Looking at the results in Table 1 one can see that after treating just twelve (12) liters of water, the commercial filter of Comparative Example 1 will have released nearly four time (4×) times the amount of silver as in Example 1 and considerably more than seven times (7×) the amount of silver as in Example 2. Assuming all three filters had the same initial amount of silver, one could foresee a markedly shorter life span for the commercial filter as compared to the inventive filters of the present invention.

### EXAMPLE 3

### COMPARATIVE EXAMPLE 4

A second set of tests was conducted comparing the long-term performance of a commercial filter, a Brita filter, and a second filter prepared using the granular activated carbon of Example 2 above. In this test, the carafes were modified with a discharge means to allow for continual, automated operation. During testing, the modified carafes were placed into an automated filling system that included sensors to determine the level of water in the filling reservoir. The sensors are connected to solenoid valves that turn the filling water off when the filling reservoir is full (1 liter) and turn the filling water back on when the filling reservoir is empty, i.e., when the full liter of water has been filtered. The system also includes in-line flow meters that indicate the total volume of water that has passed through the filter as well as means for removing effluent from the discharge means for testing. The results of this comparative study, presented as silver [Ag+] in micrograms/liter (μg/L) are shown in Table 2.

As seen with the filter of Comparative Example 1, water treated with the filter of Comparative Example 2 exceeded US EPA Safe Water Drinking Standards for the first two liters treatment passes and the World Health Organization recommended standard for, essentially, for the first seven liters of treated water. None of the samples treated with the antimicrobial activated carbon of the present invention exceeded, or even came close to exceeding, these standards. All told, the filter of Comparative Example 2 released 535.67 milligrams of silver in just the first seven (7) liters of treated water as compared to only 45.14 milligrams of silver with the filter of Example 3 made in accordance with the present invention: only about 8% of the silver of the commercial filter.

TABLE 2

| | | | Example 3 |
|---|---|---|---|
| | Comparative | | [Ag+] |
| Volume | Example 2 | Volume | (ug/L) |
| 1 | 152.8 | 1 | 7.79 |
| 2 | 108.4 | 2 | 8.149 |
| 3 | 88.78 | 3 | 7.086 |
| 5 | 72.9 | 5 | 7.382 |
| 6 | 64.13 | 6 | 7.227 |
| 7 | 48.66 | 7 | 7.503 |
| 11 | 29.2 | 8 | 7.367 |
| 22 | 16.51 | 14 | 9.658 |
| 23 | 13.17 | 15 | 9.935 |
| 30 | 11.56 | 15 | 14.39 |
| 31 | 11.32 | 19 | 16.29 |
| 32 | 11.58 | 21 | 16.63 |
| 38 | 10.59 | 27 | 19.9 |
| 42 | 9.817 | 28 | 15.78 |
| 43 | 9.415 | 28 | 14.03 |
| 49 | 10.07 | 29 | 13.88 |
| 50 | 9.597 | 30 | 13.52 |
| | | 31 | 15.58 |
| | | 40 | 16.73 |
| | | 41 | 15.21 |
| | | 42 | 13.96 |
| | | 42 | 24.24 |
| | | 43 | 19.41 |
| | | 44 | 20.75 |
| | | 65 | 16.14 |

### EXAMPLE 4 and 5

An antimicrobial thermoplastic powder was prepared by compounding 30 parts by weight of AglON AJ10D silver zeolite antimicrobial agent (2.5% silver) (AglON Technologies Inc. Wakefield, Mass.) into 70 parts by weight of Zytel® nylon (DuPont, Wilmington, Del.) The compounded material was ground to form a fine powder of approximately 500μ mean particle size. To demonstrate the versatility of the present invention, GAC was then coated with the antimicrobial nylon by two methods as follows:

In Example 4, 15.4 grams of the antimicrobial nylon powder was combined with 80 grams of a conventional GAC and dry blended at room temperature. The mixture was placed in a steel dish and the dish then placed in an oven and the oven temperature elevated to 310° C. for 55 minutes, then to 320° C. for 17 minutes and, finally, to 330° C. for 23 minutes. The dish was removed from oven and the contents stirred gently while cooling. The stirring helped smear the molten nylon across the surface of the GAC particles. Upon cooling, a fair amount of the GAC was found to have agglomerated but was readily broken up by simple hand pressure. The resultant GAC was found to have areas coated with a thin film of the antimicrobial nylon, the thickness of the nylon film ranging from about 30μ to 100μ or so.

In Example 5, 80 grams GAC was placed in a separate steel dish and subsequently placed in the same oven with the mixture of Example 4. Following the series of temperature increases and removal of Example 4 from the oven, the oven temperature was then further increased to 350° C. and the temperature held for an additional 15 minutes. The steel dish was then removed and 13.7 grams of the nylon powder containing the inorganic antimicrobial was added and blended gently. The mixture was then returned to the oven for another 10 minutes. The container was removed and blended while cooling. Again, the resultant GAC was found to have areas coated with a thin film of the antimicrobial nylon, the thickness of the nylon film ranging from about 30μ to 100μ or so, but without significant agglomeration.

Numerous characteristics and advantages have been set forth in the foregoing description, together with details of structure and function. The novel features are pointed out in the appended claims. The disclosure, however, is illustrative only, and changes may be made in detail, especially in matters of concentration, quantities and types of additives, within the principle of the invention, to the full extent indicated by the broad general meaning of the terms in which the appended claims are expressed. Further modifications of the invention herein disclosed will occur to those skilled in the respective arts and all such modifications are deemed to be within the scope of the invention as defined by the appended claims.

I claim:

1. An antimicrobial activated carbon comprising activated carbon having applied to at least a portion of its exposed outer surface an antimicrobial coating comprising a binder and an antimicrobial agent selected from the group consisting of antimicrobial soluble glass and ion-exchange type antimicrobial agents.

2. The antimicrobial activated carbon of claim 1 wherein the antimicrobial agent comprises an antimicrobial metal ion or metal ion source.

3. The antimicrobial activated carbon of claim 2 wherein the antimicrobial metal ion is selected from the group consisting of silver, copper, zinc, gold, mercury, tin, lead, iron, cobalt, nickel, manganese, arsenic, antimony, bismuth, barium, cadmium, chromium and thallium and combinations thereof.

4. The antimicrobial activated carbon of claim 1 wherein the antimicrobial agent is an antimicrobial water soluble glass comprising an antimicrobial metal ion source and a water-soluble borosilicate or phosphate glass.

5. The antimicrobial activated carbon of claim 4 wherein the antimicrobial metal ion source is the antimicrobial metal or a soluble salt thereof.

6. The antimicrobial activated carbon of claim 1 wherein the antimicrobial agent is a ion-exchange type antimicrobial agent comprising one or more ion-exchanged antimicrobial metal ions and an ion-exchange carrier therefore selected from the group consisting of zeolites, hydroxyapatites, zirconium phosphates and other ion-exchange ceramic materials.

7. The antimicrobial activated carbon of claim 6 wherein the antimicrobial metal ion is selected from the group consisting of silver, copper, zinc, and gold and combinations of any two or more of the foregoing.

8. The antimicrobial activated carbon of claim 6 wherein the antimicrobial metal ions is silver, alone or in combination with copper or zinc or both.

9. The antimicrobial activated carbon of claim 1 wherein the antimicrobial agent is a zeolite having ion-exchanged silver ions, alone or in combination with copper ions or zinc ions or both.

10. The antimicrobial activated carbon of claim 1 wherein the binder is selected from the group consisting of hydrophilic polymers, thermoset resins, thermoplastic polymer and silicates.

11. The antimicrobial activated carbon of claim 10 wherein antimicrobial coating is applied as a liquid or flowable 100% solids curable coating whose viscosity and surface tension characteristics are such that the coating will not have a tendency to remain over the pores of the activated carbon before curing.

12. The antimicrobial activated carbon of claim 1 wherein the coating is applied to no more than 60% of the exposed outer surface of the activated carbon particles.

13. The antimicrobial activated carbon of claim 1 wherein the coating is applied to no more than 50% of the exposed outer surface of the activated carbon particles.

14. The antimicrobial activated carbon of claim 1 wherein the coating is applied to no more than 40% of the exposed outer surface of the activated carbon particles.

15. The antimicrobial activated carbon of claim 1 wherein the binder is a hydrophilic polymer and the antimicrobial coating is applied to more than 60% of the exposed outer surface of the activated carbon particles.

16. The antimicrobial activated carbon of claim 15 wherein essentially the whole of the activated carbon particle is covered with the antimicrobial coating.

17. The antimicrobial activated carbon of claim 1 wherein the pressure drop across a filter made with the antimicrobial activated carbon is less than 130% of that made with the same activated carbon that is free of the antimicrobial agent.

18. The antimicrobial activated carbon of claim 1 wherein the pressure drop across a filter made with the antimicrobial activated carbon is less than 120% of that made with the same activated carbon that is free of the antimicrobial agent.

19. The antimicrobial activated carbon of claim 1 wherein the antimicrobial coating is a multi-layered coating prepared by applying multiple applications of an antimicrobial coating to the activated carbon.

20. The antimicrobial activated carbon of claim 19 wherein each successive layer of the multi-layered coating has a lower concentration of the antimicrobial agent than the preceding layer.

21. A filter comprising antimicrobial activated carbon wherein the antimicrobial activated carbon comprises activated carbon having coated on at least a portion of its exposed outer surface an antimicrobial coating comprising a binder and an antimicrobial agent selected from the group consisting of antimicrobial soluble glass and ion-exchange type antimicrobial agents.

22. The filter of claim 21 wherein the filter is a uni-body filter prepared by sintering the antimicrobial activated carbon alone or in combination with a sintering agent.

23. The filter of claim 21 wherein a sintering agent is present and comprises a thermoplastic material.

24. The filter of claim 21 wherein the antimicrobial coating is a thermoplastic coating and is present at a level sufficient to accomplish sintering of the activated carbon.

25. The filter of claim 21 which is a consumer water for in-home use in filtering potable water.

26. A method of making an antimicrobial activated carbon said method comprising the steps of applying a liquid or flowable 100% solids antimicrobial coating to at least a portion of the exposed surface of the activated carbon and curing the antimicrobial coating composition.

27. The method of claim 26 wherein the coating process is carried out on a conveyance means which carries the activated carbon through one or more spray stations which apply the antimicrobial coating to the activated carbon.

28. The method of claim 27 wherein the conveyance means carries the coated activated carbon through a curing station following the spray station.

29. The method of claim 27 wherein the coating is applied as a multi-layered coating by a plurality of successive spray stations.

30. The method of claim 29 wherein at least one of the subsequent spray stations applies a coating have a lower concentration of than the immediately preceding spray station.

31. The method of claim 29 wherein each spray station applies a coating have a lower concentration of the antimicrobial agent than the preceding spray station and, optionally, a final spray station which applies a coating free of the antimicrobial agent.

32. The method of claim 27 wherein the conveyance means is a conveyor belt.

33. The method of claim 27 wherein the conveyance means is a rotating disc.

34. The method of claim 26 wherein the coating process is carried out in a vessel having a spray nozzle for applying the antimicrobial to the activated carbon as it is being churned in the vessel.

35. The method of claim 34 wherein the vessel is a kettle type vessel with a mixer blade which chums the activated carbon concurrent with or following the application of the antimicrobial agent.

36. The method of claim 35 wherein the vessel is a rotating drum which has a nozzle which applies the antimicrobial coating to the activated carbon particles as they are churned by the rotation of the drum.

37. A method of making antimicrobial activated carbon said method comprising the step of heat fusing an antimicrobial thermoplastic powder coating material whose particle size is less than about 50μ to the exposed surface of the activated carbon.

38. The method of claim 37 wherein the activated carbon is dry blended with the antimicrobial thermoplastic powder coating material under increasing temperatures until the melt temperature of the thermoplastic powder is reached.

39. The method of claim 37 wherein the activated carbon is heated to a temperature at or above the melt temperature of the antimicrobial thermoplastic powder coating particles before the addition of the antimicrobial thermoplastic particles and then the antimicrobial thermoplastic particles are added to the heated activated carbon under mixing conditions.

40. The method of claim 37 wherein the antimicrobial thermoplastic powder coating material is added to the activated carbon material during the cool down process following the high temperature activation of the activated carbon particles but before the activated carbon particles have cooled to a temperature below the melt temperature of the antimicrobial thermoplastic powder coating particles.
